(19) United States
(12) Patent Application Publication
(10) Pub. No.: US 2004/0073154 A1
(43) Pub. Date: Apr. 15, 2004

(54) Ventricle drain

(76) Inventor: Svend Erik Borgesen, Kokkedal (DK)

Correspondence Address: FOLEY AND LARDNER, SUITE 500, 3000 K STREET NW, WASHINGTON, DC, 20007, US

(21) Appl. No.: 10/333,777

(22) PCT Filed: Jul. 25, 2001
(86) PCT No.: PCT/DK01/00517

(30) Foreign Application Priority Data
Jul. 25, 2000 (DK) PA 2000 01134
Dec. 5, 2000 (DK) PA 2000 01824

(51) Int. Cl. A61M 5/00; A61M 27/00

(52) U.S. Cl. 604/8; 604/541

(57) ABSTRACT

The invention provides means by which access to the intracranial compartment is completely sealed from the skin and the scalp tissues, which makes it possible to change the drain when necessary without bringing the drain in the contact with the scalp. It also provides an improved ventricle draining system supporting a better fixation of the drain to avoid the drain from sliding. The drain comprises a fixture with a conduit defining passage through the fixture, the fixture being provided with fastening means for attachment of the fixture to an aperture in the cranial bone, a seal for sealed engagement with a catheter inserted in the fixture, and fastener for securing the catheter to the fixture.

### FIELD OF THE INVENTION

The present invention relates to a cerebrospinal fluid draining system for draining cerebrospinal fluid from the brain ventricles through a sealed passage in a scalp aperture.

### DESCRIPTION OF THE PRIOR ART

External draining of cerebrospinal fluid (CSF) from the intracranial CSF compartments is a standard neuro-surgical method aiming at reducing increased intracranial pressure. CSF is formed in the ventricular system irrespective of the intracranial pressure (ICP). The formation rate is constant, with a range of 0.3-0.4 ml/min. (Borgesen and Gjerris 1987). Increased intracranial pressure, arise when the outflow of the CSF is obstructed, eventually leading to an increase in the amount of intracranially located cerebrospinal fluid. The obstruction may be localised in the aqueduct or the IV ventricle or in the normal resorption sites in villi arachnoidales in connection with the sagittal sinus.

Typically a passage is provided in the cranial bone, e.g. by means of a hole drilled through the skull. A catheter drain is thereafter inserted through the passage and into the ventricular system. The catheter is secured to the skin by means of an adhesive. The drain may be led via a subcutaneous duct in order to avoid a clinical contamination wall via the skin perforation for the drain and the hole in the cranial bone, with the exposed intracranial content directly beneath.

Clinical experience has proven that this principle of external CSF draining is not an ideal solution. The often confused and motor hyperactive patient unintentionally removes the ventricular drain by simply pulling it out of the scalp. The drain can easily slide in and out through the cutaneous canal which increases the risk of infection. Reinsertion of the ventricular drain through the same skin opening further increases the risk of infection.

The risk of infection depends very much on the duration of the period where draining is needed. On average 5-10% of all patients (irrespectively of the length of drainage) develop infection of the cerebrospinal fluid. In many neuro-surgical units, the experience is that drainage for more than 5 days results in infection in more than 90% of the cases. While infection of the CSF in most cases can be treated by antibiotics, infection increases the length of the hospitalisation—typically in intensive care units—and adds morbidity following the initial disease.

### DESCRIPTION OF THE INVENTION

An object of the present invention is to provide means by which access to the intracranial compartment is completely sealed from the skin and the scalp tissues which makes it possible to change the drain when necessary and without bringing the drain in contact with the scalp. It is a further object of the invention to provide an improved ventricle draining system supporting a better fixation of the drain to avoid the drain from sliding.

Accordingly, a first aspect of the present invention relates to a ventricle drain comprising:

sealing means for providing a sealed passage through an aperture in a cranial bone, and a catheter having a free end and an end adapted for insertion into the aperture through the sealed passage for draining bodily fluids,

wherein the sealing means comprises

a fixture with a conduit defining a passage through the fixture, the fixture being provided with fastening means for attachment of the fixture to the aperture in the cranial bone,

a seal for sealed engagement with the catheter and the fixture, and

a fastener for securing the catheter to the fixture.

The invention thus provides means for passing a catheter through a sealed passage through a fixture attached to the aperture of the scull. The fixture could be attached to the scull as the aperture is drilled, e.g. when the treatment of the patient begins. The bone and skin tissue will grow into a sealing engagement with the fixture and since the fixture is not to be replaced, the risk of infections occurring between the fixture and the skull is low. By means of the fixture and the fastener, the catheter and/or the seal may be repositioned or replaced repeatedly without interfering with the skin and bone tissue of the scull.

The fixture may have any cross sectional shape. However, according to a preferred embodiment of the invention, the cross sectional shape of at least a part of the fixture is circular. The circular shape enables the fixture to be fastened to a drilled hole in the scull by means of a threaded joint or screw joint. The hole in the scull could e.g. be provided with an internal thread wherein external threads of the fixture are screwed by axial rotation of the fixture. The external threads of the fixture may also be sharpened so that the threads cut a corresponding track in the skull when the fixture is screwed into the skull. The fixture may also be attached to the scull by means of other gripping arrangements, e.g. by means of a number of small hooks engaging in the cranium or by means of a plug being inserted into the aperture and being expanded, e.g. by insertion of a conical mandrel into a cavity of the plug.

The fixture has a conduit or passage for passing the catheter through the skull. The passage could be divided into a proximal (lower) part and a distal (upper) part, the proximal part defining the inserted end of the fixture and thus being closest to the patient, when fitted in an aperture. If an intermediate part of the passage, between the proximal part and the distal part, is provided with a smaller radial size, the seal may be prevented from passing through the passage. According to one embodiment, the radial size of the proximal part of the passage is smaller than the radial size of the proximal part of the passage. Thereby, the transition between the proximal part and the distal part of the passage provides a seat for the seal. According to another embodiment, the passage is divided into a proximal part and a distal part by an intermediate part, which intermediate part being provided with a smaller radial size than at least the distal part and optionally also with a smaller radial size than the proximal part. The seat for the seal can also be established by inserting a locking washer into the passage. The passage could e.g. be provided with an internal circular groove for catching a washer. The division of the passage in two parts, either by use of a washer or by means of changing radial size of the passage, prevents the seal from moving down through the fixture and into the brain.

According to a preferred embodiment of the invention, the passage through the fixture is provided with two end parts and an intermediate part. The two end parts being provided with radial sizes which are larger than the radial size of the intermediate part. As an example the fixture may be constricted by the two end parts being provided with decreasing radial sizes towards the intermediate part of the passage. In that way, the catheter may easily be positioned with guiding means such as an introducer or stylet—e.g. a stiff needle—within a circular area defined by the limitations of the introducer and catheter to move in the passage. The wider the end portions are the better a manoeuvrability of the guiding means is achieved. When the catheter has been positioned in the brain, the guiding pin is removed and the fastener is attached for locking the catheter tightly to the fixture.

The fastening means of the fastener may comprise threads for establishment of a screw joint between the fastener and the fixture, e.g. by axial rotation of the fastener. The fastener may be provided with either external or internal threads corresponding to respectively internal or external threads of the fixture. The fastener could also be attached to the fixture by other fastening means such as by means of a snap locking-arrangement.

The seal is preferably adapted to have at least a first and a second shape corresponding to a first and a second position of the fastener in relation to the seal, and wherein at least the first position provides a sealed engagement between the seal and the catheter. The seal should be adapted between the catheter and the fixture. The two shapes of the seal could be provided e.g. by means of a flexible seal. As an example, the edges of the seal may deflect due to the pressure between the fixture and the fastener. The seal may also have one or more deflectable edges which, upon the pressure between the fastener and the fixture, bends into engagement with the catheter. The catheter should be capable of resisting this radially directed pressure from the seal. The catheter could therefore be provided with an area being reinforced against radial pressure. The reinforcement could be provided at least in a part of the length of the catheter. The reinforcement could be achieved e.g. by inserting a tubular body into the catheter or by inserting the catheter into a tubular body. The catheter may simply be made from a material which is either strong enough to resist the pressure or which alternatively can be hardened in the surface so as to resist the pressure from the seal. According to a preferred embodiment the catheter is made in composition of at least two materials having different surface hardness or strength and/or resiliency.

According to a preferred embodiment, the drain further comprises a valve with at least a first port attached to the free end of the catheter and a second port attached to a place of disposal of the bodily fluids or to a resorption site of the body. By means of the valve or by means of an additionally mounted one-way valve, it must be ensured that the fluids do not run back through the catheter and into the brain again. Preferably the valve also has at least a third port and a switch for selectively connecting one of either the second port or the third port to the first port. In that way, the catheter may selectively be connected to the place of disposal or to a free additional port. One more additional fourth port could be arranged along the centre line of the catheter thus allowing a guiding pin to be inserted into the catheter. A guiding pin is useful when the catheter is to be positioned in the brain. An additional port could also be useful for taking samples from the fluid. The port could be provided e.g. with a soft rubber closure which is penetrable by a needle and soft enough to close tightly when the needle is removed—a so called puncture.

According to a second aspect, the present invention relates to a method for passing a catheter though a aperture in the cranial bone, said method comprising the steps of:

attaching a fixture provided with a conduit to the cranial bone,

inserting the catheter into the brain through the conduit, and

fixating the catheter between the fixture and a seal,

said seal being compressed into sealing engagement with the catheter and the fixture by a fastener attached to the fixture.

### DETAILED DESCRIPTION OF THE INVENTION

A preferred embodiment of the invention will now be described in details with reference to the drawing in which:

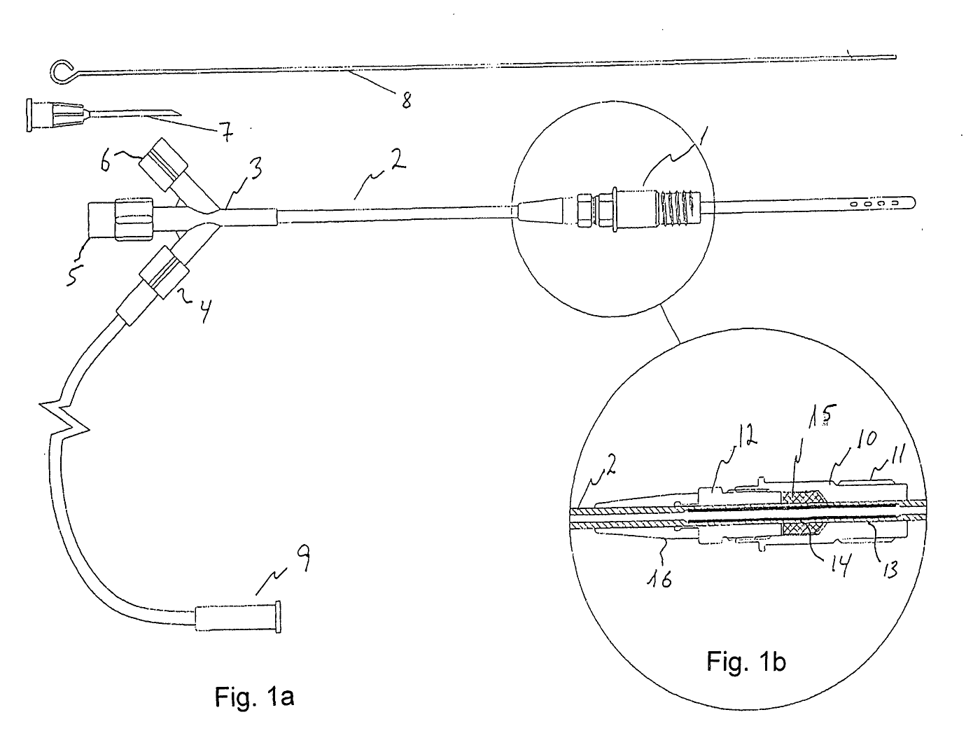

FIG. 1a shows a ventricle drain system according to the present invention,

FIG. 1b shows an enlarged view of a sealed passage for the drain system,

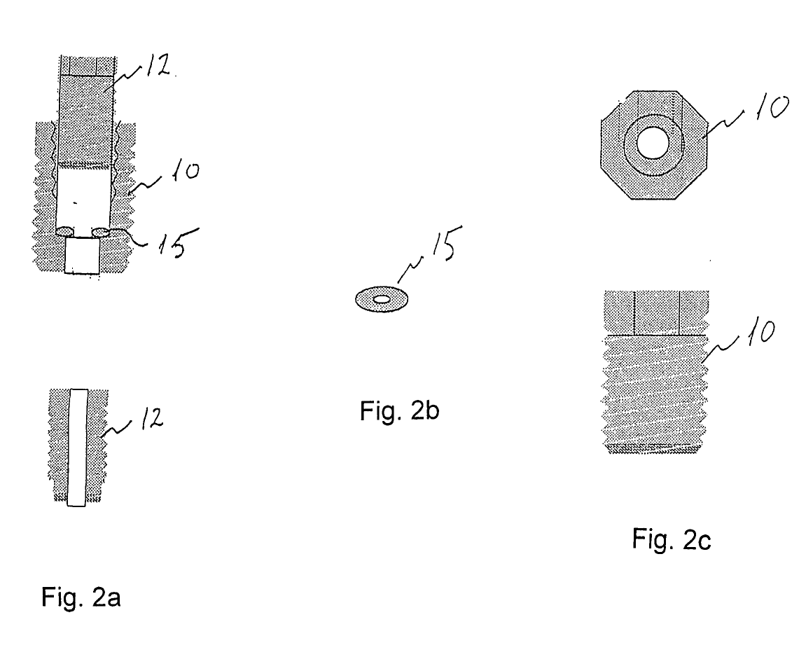

FIG. 2a shows a cross sectional view of a bolt connection providing a sealed passage,

FIG. 2b shows a seal for sealing the passage,

FIG. 2c shows a fixture for attachment of the sealed passage to a scull aperture,

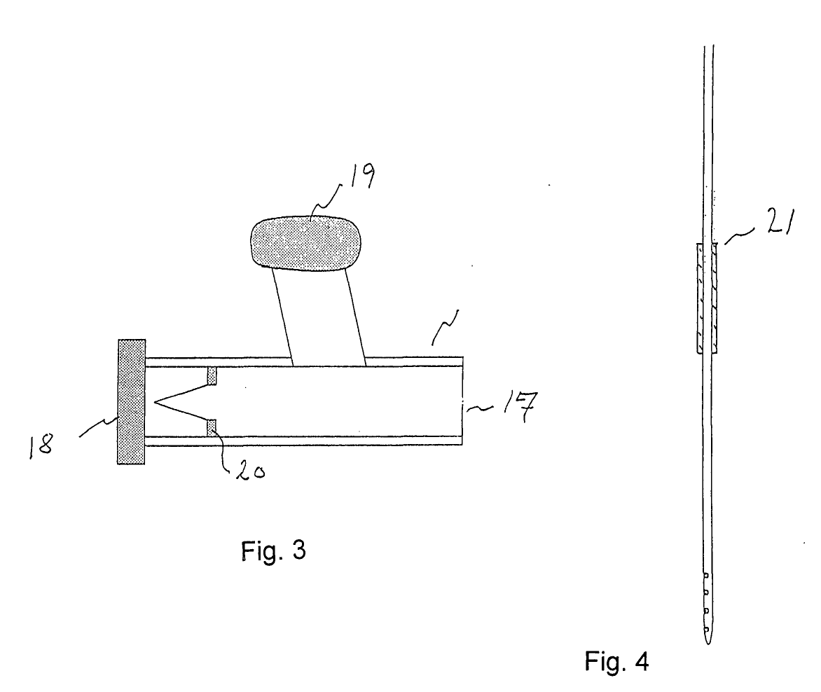

FIG. 3 shows a catheter connector with a one-way valve,

FIG. 4 shows a catheter for draining fluids through the sealed passage,

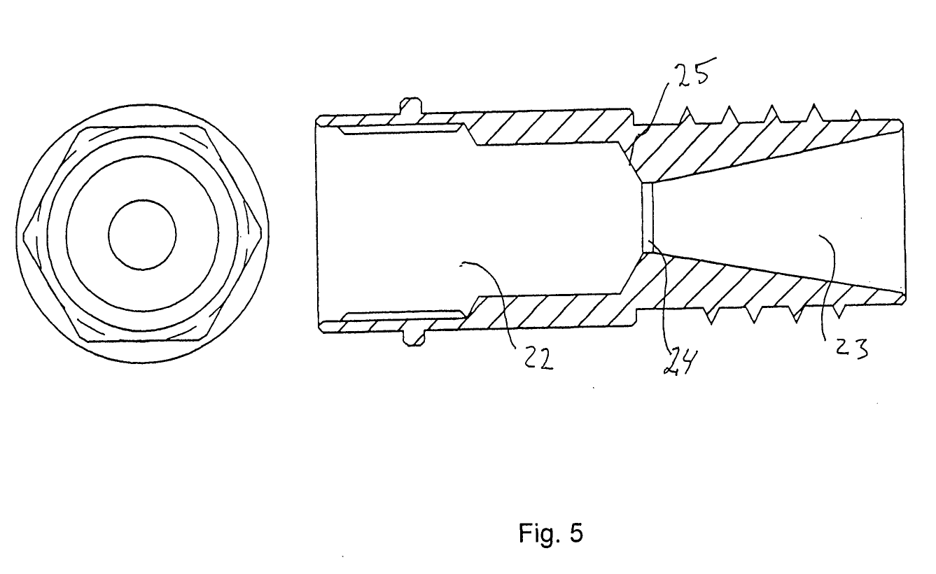

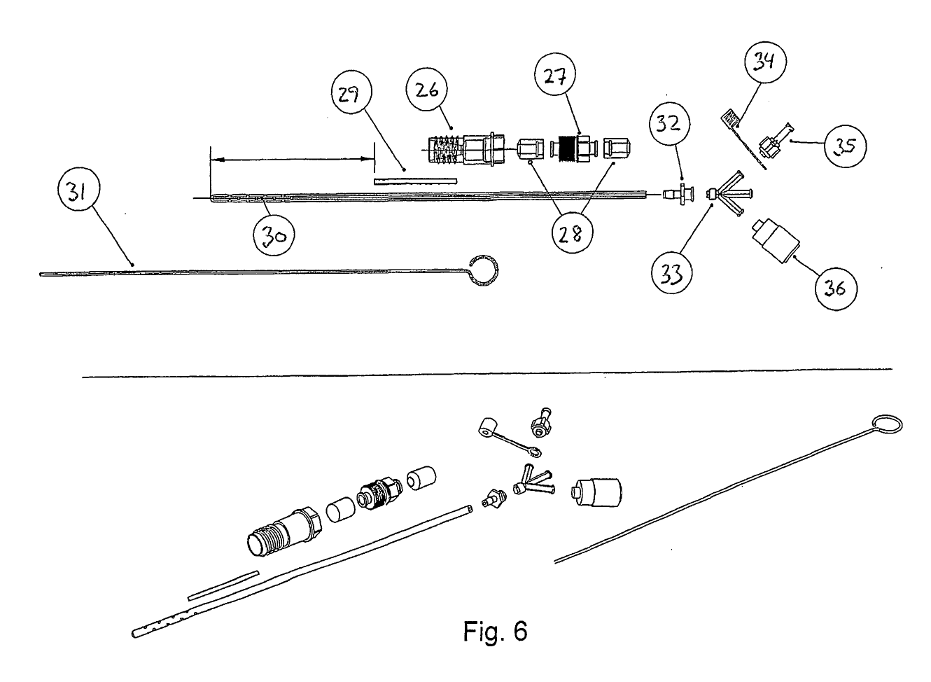

FIG. 5 shows an alternative embodiment of the fixture, and

FIG. 6 shows a drawing of drain, the drawing referencing table 1 of the following detailed description.

Referring to FIG. 1 the ventricle drain system has a bolt connection 1 to be fastened in an aperture of a scull. The bolt 1 provides a sealed passage for the one end of the catheter 2 through the scull. The other end of the catheter 2 is connected to a connector 3. In the shown example, the connector is a 4 ways-valve by means of which the catheter selectively can be connected to one of the three ports 4,5,6. The outlet port 4 is adapted for connection of the catheter to a place of disposal.

The place of disposal could be a drainage bag. The port 5 is arranged along the centre line of the catheter and is thus adapted for insertion of guiding means such as the pin 8 for positioning of the catheter in a specific section of the brain. The port 6 could be provided with a rubber seal or puncture, which can be punctured by the needle 7, e.g. for taking samples of the fluid.

Referring to FIG. 1b, the bolt connection 1 has a fixture 10 with a threaded section 11 adapted to be screwed into a hole in a skull. The fixture 10 has a channel 13 through which channel the catheter 2 can be passed. The seal 15, being compressed by the fastener 12 screwed into the fixture 10, seals the channel. The compression of the seal leads to a radial expansion of the seal. This compression again leads to a sealing compression between the seal and respectively the fixture 10 and the catheter 2 which again locks the catheter in its position relative to the fixture 10 and thus relative to the skull.

In order to support the catheter against the compression, the catheter may have a reinforced part 14, e.g. reinforced by the insertion of a tubular body into the catheter. Due to the flexibility and thus the ability of positioning the catheter in the brain the reinforced part should preferably be limited to a length of approximately the size of the bolt connection 1 and it should not extend into the brain.

The support hose 16 supports the catheter in order to avoid a too sharp bending of the catheter. A sharp bending of the catheter may lead to a collapse of the catheter whereby the passage through the catheter is cut off.

As best seen in to FIG. 2a the fastener 12 has threads for fastening the fastener into the fixture 10. The fastener 12 further has a channel through which the catheter can be passed.

The seal 15 could preferably be made of a resilient material such as silicone rubber, which easily deforms elastically upon the pressure from the fixture and the fastener 10,12.

The seal could alternatively be made with a number of flexibly mounted cutting edges which, upon the pressure of the fixture and the fastener, cuts into the catheter. The seal could also be made of a material that deforms in-elastically e.g. a soft metal such as brass or aluminium, which upon the pressure of the fixture and the fastener deflects and engages the catheter.

Both the fixture and the fastener 10,12 could preferably be provided with a square or hexagonal head 16,17 for an easy fastening of the catheter using a bracket.

As an alternative to the threaded engagement between the fastener and the fixture, the fastener 12 could also be attached by means of a snap-fastener arrangement. The snap-fastener arrangement could be provided by means of a projection on one part of either the fastener or the fixture. The projection being adapted for engagement with embossing or depression marks in the other part of either the fastener or the fixture when the fastener is pressed onto the fixture. The snap-fastener arrangement thus enables an easy and fast fixation and sealing of the catheter to the fixture without using any tools. The snap-fastener arrangement further provides the advantage that the same predetermined pressure always is applied to the seal.

The fixture and the fastener 10,12 could be made of any material with a low resiliency relative to the catheter and with a resistance against the fluids, e.g. stainless steel, titanium, a ceramic material or composite material with ceramic or plastic or it could be made of plastic. The fixture may be provided with a soft rubber seal arranged between the fixture and the scull or the threads of the fixture may be coated with a sealing material such as Teflon. The Teflon could either be sprayed onto the surface of the threaded end of the fixture or taped onto the threaded end of the fixture.

FIG. 3 shows another example of a connector for connecting the catheter e.g. to a draining bag. The connector has a port 17 for the catheter, a port 18 for a draining bag etc. and a port 19 e.g. for using a needle to take samples through a soft rubber seal. The connector further comprises a one-way valve 20. The one-way valve is adapted in order to secure that fluids from the drainage bag can not flow back through the catheter and into the brain. The one way valve or the entire connector could be mounted in addition to the connector 3 shown in FIG. 1, e.g. between the draining bag and the connector. Under all circumstances a one-way valve should be inserted between the draining bag and the catheter.

Referring to FIG. 4 another embodiment of the catheter may have a reinforced part 21, provided around the exterior surface of the catheter. The reinforced part could e.g. be a tubular element made of a material with a low resiliency compared with the catheter, e.g. a tubular piece of metal or hard plastic glued to the catheter. The reinforced part could also be a section of the tubular catheter in it self being more resistant to the pressure from the seal than the rest of the catheter.

FIG. 5 shows an alternative embodiment of the fixture. The passage of the fixture is provided with two end parts 22,23 and an intermediate part 24. The radial size of the intermediate part is smaller than the radial size of the two end parts. The end part 23 of the passage, the part being closest to the brain, is provided with a conical shape having the largest radial size nearest the brain. The shape of the two end parts enables manoeuvrability in the positioning of the catheter by means of the guiding pin 8. The intermediate part 24 and the surface 25 serve as a seat for the seal.

The following table describes preferred materials for the individual parts of the drain, with reference to FIG. 6.

TABLE NO 1

| | | Chemical | | Ref. no |
|---|---|---|---|---|
| Component | Trade Name | Name | Supplier | in FIG. 6 |
| Glue | Nusil MED- | | Nusil - USA | |
| | 1511 | | | |
| Hollow skull | Titanium | Grade | Soltech - | 26 |
| screw (fixture) | | 2 TI | Denmark | |
| Duckbill valve | Silicone | | Rodia | |
| | LSR 40 | | Silicones-USA | |
| Hollow adapter | Titanium | Grade | Soltech - | 27 |
| screw (fastener) | | 2 TI | Denmark | |
| Draintube | Silicone | | Rodia | 28 |
| gasket | LSR 60 | | Silicones-USA | |
| Titan Tube | Titanium | Grade | Soltech - | 29 |
| | | 2 TI | Denmark | |
| Silicone | Nusil MED | | Nusil - USA | 30 |
| ventricle drain | 4750 loaded | | | |
| | with 10% | | | |
| | BAS04 | | | |
| 4-Port Y | ABS Natural | | Promepla - | 33 |
| connection | Poly- | | Monaco | |
| | propylene | | | |
| Connection for | Poly- | | Promepla - | 32 |
| tubing | carbonate | | Monaco | |
| Cap | Poly- | | Promepla - | 34 |
| | ethylene | | Monaco | |
| Injection port | Polyisoprene | | Promepla - | 36 |
| | ABS natural | | Monaco | |
| Stylet | Clinical/ | | Soltech - | 31 |
| | surgical | | Denmark | |
| | steel 304 | | | |

1. A ventricle drain comprising:
sealing means for providing a sealed passage through an aperture in a cranial bone, and a catheter having a free end and an end adapted for insertion into the aperture through the sealed passage for draining bodily fluids,
wherein the sealing means comprises
a fixture with a conduit defining a passage through the fixture, the fixture being provided with fastening means for attachment of the fixture to the aperture,
a seal for sealed engagement with the catheter and the fixture, and
a fastener for securing the catheter to the fixture.

2. A ventricle drain according to claim 1, wherein the cross sectional shape of at least a part of the fixture is circular and wherein the fastening means of the fixture comprises threads for establishment of a screw joint between the aperture and the fixture by axial rotation of the fixture.

3. A ventricle drain according to claim 1 or 2, wherein the passage through the fixture is provided with two end parts and an intermediate part and wherein:
the two end parts are provided with a first and a second radial size, and
the intermediate part of the passage is provided with a third radial size,
and wherein the third radial size is smaller than the first and the second radial sizes.

4. A ventricle drain according to claim 3, wherein the passage through the fixture is constricted by the two end parts being provided with decreasing radial sizes towards the intermediate part of the passage.

5. A ventricle drain according to any of the preceding claims, wherein the fastening means of the fastener comprises threads for establishment of a screw joint between the fastener and the fixture by axial rotation of the fastener.

6. A ventricle drain according to any of the preceding claims, wherein the seal is adapted to have at least a first and a second shape corresponding to a first and a second position of the fastener in relation to the seal, and wherein at least the first position provides a sealed engagement between the seal and the catheter.

7. A ventricle drain according to any of the preceding claims, wherein the catheter is reinforced against radial pressure at least in a part of its length.

8. A ventricle drain according to any of the preceding claims, wherein the catheter is reinforced by means of a body inserted into the catheter.

9. A ventricle drain according to any of the preceding claims, further comprising a valve having a first port attached to the free end of the catheter, a second port attached to a place of disposal of the bodily fluids and a third port, said valve having means for selectively connecting one of either the second port or the third port to the first port.

10. A ventricle drain according to claim 9, wherein the third port is adapted for insertion of guiding means for guiding the catheter through the aperture.

11. A ventricle drain according to claim 9 or 10, further comprising a one-way valve between the second port and the place of disposal so as to avoid the bodily fluids to flow from the place of disposal to the catheter.

12. A ventricle drain according to any of claims 9-11, wherein the third port is adapted for injection of fluids into the free end of the catheter.

13. A ventricle drain according to any of claims 9-11, wherein the valve further comprises a fourth port with a soft rubber seal adapted for injection of fluids into the free end of the catheter.

14. A catheter for a ventricle drain according to any of the preceding claims, wherein the catheter has an intermediate part and two end parts, the intermediate part of the catheter being made from a different material than the end parts, so that the intermediate part of the catheter is more resistant towards radial pressure than the end parts.

15. A seal for a ventricle drain according to any of the preceding claims, wherein the seal deforms elastically in a radial direction so as to press against the catheter upon application of a pressure to the seal.

16. A seal for a ventricle drain according to any of the preceding claims, wherein the seal has a first and a second part the first part of the seal being provided with a resilient part enabling the first part to deform elastically in a radial direction so as to press against the catheter upon application of an axial pressure to the seal.

17. A method for passing a catheter though a aperture in the cranial bone, said method comprising the steps of:
attaching a fixture provided with a conduit to the cranial bone,
inserting the catheter into the brain through the conduit, and
fixating the catheter between the fixture and a seal,
said seal being compressed into sealing engagement with the catheter and the fixture by a fastener attached to the fixture.
